(19) United States
(12) Patent Application Publication
(10) Pub. No.: US 2008/0171038 A1
(43) Pub. Date: Jul. 17, 2008

(54) METHODS OF TREATING IDIOPATHIC THROMBOCYTOPENIA PURPURA USING A GM-CSF ANTAGONIST

(75) Inventors: Christopher R. Bebbington, San Mateo, CA (US); Geoffrey T. Yarranton, Burlingame, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KaloBios Pharmaceuticals, Inc., Palo Alto, CA (US)

(21) Appl. No.: 11/937,179

(22) Filed: Nov. 8, 2007

Related U.S. Application Data
(60) Provisional application No. 60/902,741, filed on Feb. 21, 2007.
(60) Provisional application No. 60/858,258, filed on Nov. 8, 2006.

(51) Int. Cl. A61K 39/395; A61P 7/00

(52) U.S. Cl. 424/133.1; 424/130.1; 424/143.1; 424/178.1

(57) ABSTRACT

The invention is based on the discovery that GM-CSF antagonists can be used for the treatment of Idiopathic Thrombocytopenia Purpura (ITP). Accordingly, the invention provides methods of administering a GM-CSF antagonist, e.g., a GM-CSF antibody, to a patient that has ITP and pharmaceutical compositions comprising such antagonists.

### CROSS-REFERENCE TO RELATED APPLICATIONS

This application claims benefit of U.S. provisional application No. 60/858,258, filed Nov. 8, 2006 and U.S. provisional application No. 60/902,741, filed Feb. 21, 2007, each of which applications is herein incorporated by reference.

### BACKGROUND OF THE INVENTION

Idiopathic thrombocytopenia purpura (ITP) is a hematologic disorder defined clinically by low platelet count, normal bone marrow and the absence of other causes of thrombocytopenia. The disorder is believed to be due to an autoimmune disease in which production of auto-antibodies (usually IgG) leads to excessive depletion of platelets. Consequently, the disease is also referred to as immune thrombocytopenia or autoimmune thrombocytopenia (AITP). The etiology is not understood and the antigen or antigens which initiate and sustain the autoimmune response are not known. Patients typically present with petechiae or purpura that develop over several days, accompanied by platelet counts less than 20,000×10⁹/L. Severe cutaneous bleeding, prolonged epistaxis, gingival bleeding, overt hematuria, or menorrhagia may develop at platelet counts less than 10,000×10⁹/L. Spontaneous or posttraumatic ICH or bleeding at other internal sites is uncommon, but not without precedence, at platelet counts between 10,000 and 20,000×10⁹/L. Those with platelet counts between 30,000 and 50,000×10⁹/L may note easy bruising, whereas platelet counts above 50,000×10⁹/L are usually discovered incidentally. Rarely do patients present with bleeding disproportionate to the platelet count because of antibody-induced platelet dysfunction. Some patients experience untoward and otherwise unexplained fatigue when their platelet count is low.

ITP is poorly treated at present. Corticosteroids, such as methylprednisolone or dexamethasone, are given to suppress the immune response. A high proportion of patients with chronic ITP are refractory to corticosteroids, in which case intravenous immune globulins (IVIG) or splenectomy are typically evaluated. Other drugs such as vincristine, azathioprine (Imuran), Danazol, cyclophosphamide, and cyclosporine are prescribed for patients in severe cases that are refractory to other treatments. Further treatments that have been evaluated for use in the treatment of ITP include the anti-CD20 antibody rituximab, anti-CD16 antibody, and anti-CD154 antibody.

IVIG has been shown to be effective in clinical studies for the treatment of ITP (Imbach et al (1981) Lancet, 1981, 1:128-1231) and is approved for use in the treatment of the disease in the United States and other countries. The mechanism of action of IVIG preparations is not well understood, however. A number of activities have been ascribed to IVIG including modulation of complement activation products, suppressing idiotypic antibody, saturating Fc receptors on macrophages, and suppressing various inflammatory mediators including cytokines, chemokines, and metalloproteinases. Each of these activities could play a role in modulation of the immune system and in amelioration of ITP.

Preparations of IVIG have been reported to contain detectable levels of antibodies to human cytokines and it has been speculated that such antibodies may contribute to IVIG activity. For example antibodies to TNF-alpha and TNF-beta (Lymphotoxin) have been found in normal human immunoglobulin preparations (Jeffes et al (1989) Arth. Rheum. 32: 1148) and antibodies to GM-CSF have also been found in IVIG. However, the levels of such antibodies vary between different preparations of IVIG.

Intravenous Anti-D antibodies are also used in the treatment of ITP in Rh+ individuals and these may be combined with IVIG therapy. The mechanism of action of these agents is not understood, but it is thought that they block Fc-mediated platelet destruction and/or clearance. After treatment of juvenile ITP patients with Anti-D antibodies, serum levels of pro-inflammatory cytokines including IL-6, TNF and GM-CSF, have been shown to rise and it has been suggested that this induction of a “cytokine storm” by Anti-D antibodies may contribute to the therapeutic effect in ITP by stimulating the reticulo-endothelial system to induce clearance of red blood cells and inhibit the clearance of platelets (Semple et al (2002) Am. J. Hematol. 69: 225). GM-CSF levels have been shown to be elevated in ITP patients (Nomura et al 1995; Abboud et al (1996) Br. J. Haematol. 92: 486) and in a study of childhood ITP (Rizk et al (2004) Med Sci Monitor 10: CR330). Aboud et al. speculated that GM-CSF may play a role in the body's response to thrombocytopenia, whereas Rizk et al. suggested that GM-CSF may play a pathological role. Thus, the mechanism of action of intravenous immunoglobulin administration in the treatment of ITP and the potential role played by GM-CSF and other cytokines remains controversial.

Antibodies that antagonize the activity of GM-CSF are known in the art. For example, U.S. Pat. No. 6,720,155 describes antibodies that antagonize the activity of GM-CSF, IL-3 or IL-5 (cytokines which share a common receptor sub-unit). This patent describes the use of such anti-receptor antibodies for the treatment of rheumatoid arthritis and possibly other inflammatory conditions. Furthermore, US Pat. Pub. No. 20040053365 describes chimerized antibodies to GM-CSF and their use for the treatment of “inflammatory conditions” including autoimmune disorders such as psoriasis, rheumatoid arthritis, lupus, post-ischemic leukocyte mediated tissue damage (reperfusion injury), frost-bite injury or shock, acute leukocyte-mediated lung injury (acute respiratory distress syndrome or ARDS), asthma, traumatic shock, septic shock, nephritis, acute and chronic inflammation, and platelet-mediated pathologies such as arteriosclerosis and inappropriate blood clotting.

The administration of GM-CSF antagonists, as described herein, addresses the need for alternative ITP treatments.

### BRIEF SUMMARY OF THE INVENTION

The present invention is based on the discovery that a GM-CSF antagonist can be used to treat ITP. Thus, the invention provides methods of administering a GM-CSF antagonist, e.g., an antibody, to a patient that has ITP with the proviso that the antagonist is not human IVIG. In some embodiments, the GM-CSF antagonist is recombinantly produced, e.g., a recombinant monoclonal antibody. In other embodiments, the GM-CSF antagonist, e.g., purified anti-GM-CSF from human plasma, is purified from a natural source.

In one aspect, the invention provides a method for treating a patient suffering from idiopathic thrombocytopenia purpura (ITP), the method comprising administering a therapeutically effective amount of a purified GM-CSF antagonist to the patient in an amount sufficient to reduce the symptoms of ITP. A GM-CSF antagonist can be e.g., an anti-GM-CSF antibody, an anti-GM-CSF receptor antibody; a soluble GM-CSF receptor; a cytochrome b562 antibody mimetic; an adnectin, a lipocalin scaffold antibody mimetic; a calixarene antibody mimetic, or an antibody like binding peptidomimetic.

In many embodiments, the GM-CSF antagonist is an antibody to GM-CSF, i.e., an anti-GM-CSF antibody. In various embodiments, the antibody can be a polyclonal antibody, a monoclonal antibody, or an antibody such as a nanobody or a camelid antibody. In some embodiments, the antibody is an antibody fragment, such as a Fab, a Fab′, a F(ab′)₂, a scFv, or a domain antibody (dAB). The antibody can also be modified, e.g., to enhance stability. Thus, in some embodiments, the antibody is conjugated to polyethylene glycol.

In some embodiments, the antibody has an affinity of about 100 pM to about 10 nM, e.g., from about 100 pM, about 200 pM, about 300 pM, about 400 pM, about 500 pM, about 600 pM, about 700 pM, about 800 pM, about 900 pM, or about 1 nM to about 10 nM. In further embodiments, the antibody has an affinity of about 1 pM to about 100 pM, e.g., an affinity of about 1 pM, about 5 pM, about 10 pM, about 15 pM, about 20 pM, about 25 pM, about 30 pM, about 40 pM, about 50 pM, about 60 pM, about 70 pM, about 80 pM, or about 90 pM to about 100 pM. In some embodiments, the antibody has an affinity of from about 10 to about 30 pM.

In some embodiments, the antibody is a neutralizing antibody. In further embodiments, the antibody is a recombinant or chimeric antibody. In some embodiments, the antibody is a human antibody. In some embodiments, the antibody comprises a human variable region. In some embodiments, the antibody comprises a human light chain constant region. In some embodiments, the antibody comprises a human heavy chain constant region, such as a gamma chain.

In further embodiments, the antibody binds to the same epitope as a chimeric 19/2 antibody. The antibody can, e.g., comprise the V_{H} and V_{L} regions of chimeric 19/2. The antibody can also comprise a human heavy chain constant region such as a gamma region. In some embodiments, the antibody comprises the CDR1, CDR2, and CDR3 of the V_{H} region of chimeric 19/2. In further embodiments, the antibody comprises the CDR1, CDR2, and CDR3 of the V_{L} region of chimeric 19/2. In additional embodiments, the antibody comprises the CDR1, CDR2, and CDR3 of the V_{H} and V_{L} regions of a chimeric 19/2 antibody. In some embodiments, the antibody comprises the V_{H} region CDR3 and V_{L} region CDR3 of chimeric 19/2.

In some embodiments, the antibody has a half-life of about 7 to about 25 days.

In some embodiments of the methods of the invention, the GM-CSF antagonist, e.g., an anti-GMCSF antibody, is administered by injection or by infusion. For example, the GM-CSF antagonist can be administered intravenously over a period between about 15 minutes and about 2 hours.

In other embodiments, the GM-CSF antagonist is administered subcutaneously by bolus injection.

In further embodiments, the GM-CSF antagonist is administered intramuscularly.

A GM-CSF antibody can, for example, be administered at a dose between about 1 mg/kg of body weight and about 10 mg/kg of body weight.

In some embodiments, treatment with the GM-CSF antagonist comprises a second administration of the GM-CSF antagonist.

In some embodiments, the treatment methods of the invention further comprise administering a second ITP therapeutic agent, such as a corticosteroid (e.g., methyl prednisolone or dexamethasone), IVIG, or Anti-D.

### DETAILED DESCRIPTION OF THE INVENTION

### I. Definitions

As used herein, “idiopathic thrombocytopenia purpura (ITP)” refers to a hematologic disorder defined clinically by low platelet count, typically less than 50,000×10⁹/L, normal bone marrow and the absence of other causes of thrombocytopenia.

As used herein, “Granulocyte Macrophage-Colony Stimulating Factor” (GM-CSF) refers to a small a naturally occurring glycoprotein with internal disulfide bonds having a molecular weight of approximately 23 kDa. In humans, it is encoded by a gene located within the cytokine cluster on human chromosome 5. The sequence of the human gene and protein are known. The protein has an N-terminal signal sequence, and a C-terminal receptor binding domain (Rasko and Gough In: The Cytokine Handbook, A. Thomson, et al, Academic Press, New York (1994) pages 349-369). Its three-dimensional structure is similar to that of the interleukins, although the amino acid sequences are not similar. GM-CSF is produced in response to a number of inflammatory mediators by mesenchymal cells present in the hemopoietic environment and at peripheral sites of inflammation. GM-CSF is able to stimulate the production of neutrophilic granulocytes, macrophages, and mixed granulocyte-macrophage colonies from bone marrow cells and can stimulate the formation of eosinophil colonies from fetal liver progenitor cells. GM-CSF can also stimulate some functional activities in mature granulocytes and macrophages.

The term “granulocyte macrophage-colony stimulating factor receptor” (GM-CSFR)” refers to a membrane bound receptor expressed on cells that transduces a signal when bound to granulocyte macrophage colony-stimulating factor (GM-CSF). GM-CSFR consists of a ligand-specific low-affinity binding chain (GM-CSFR alpha) and a second chain that is required for high-affinity binding and signal transduction. This second chain is shared by the ligand-specific alpha-chains for the interleukin 3 (IL-3) and IL-5 receptors and is therefore called beta common (beta c). The cytoplasmic region of GM-CSFR alpha consists of a membrane-proximal conserved region shared by the alpha 1 and alpha 2 isoforms and a C-terminal variable region that is divergent between alpha 1 and alpha 2. The cytoplasmic region of beta-c contains membrane proximal serine and acidic domains that are important for the proliferative response induced by GM-CSF

The term “soluble granulocyte macrophage-colony stimulating factor receptor” (sGM-CSFR) refers to a non-membrane bound receptor that binds GM-CSF, but does not transduce a signal when bound to the ligand.

As used herein, “GM-CSF antagonist” refers to a molecule or compound that interacts with GM-CSF, or its receptor, to reduce or block (either partially or completely) signal transduction that would otherwise result from the binding of GM-CSF to its cognate receptor expressed on cells. GM-CSF antagonists may act by reducing the amount of GM-CSF ligand available to bind the receptor (e.g., antibodies that once bound to GM-CSF increase the clearance rate of GM-CSF) or prevent the ligand from binding to its receptor either by binding to GM-CSF or the receptor (e.g., neutralizing antibodies). GM-CSF antagonist may also include inhibitors, which may include compounds that bind GM-CSF or its receptor to partially or completely inhibit signaling. GM-CSF antagonist may include antibodies, natural or synthetic ligands or fragments thereof, polypeptides, small molecules, and the like.

A “purified” GM-CSF antagonist as used herein refers to a GM-CSF antagonist that is substantially or essentially free from components that normally accompany it as found in its native state. For example, a GM-CSF antagonist such as an anti-GM-CSF antibody, that is purified from blood or plasma is substantially free of other blood or plasma components such as other immunoglobulin molecules. Purity and homogeneity are typically determined using analytical chemistry techniques such as polyacrylamide gel electrophoresis or high performance liquid chromatography. A protein that is the predominant species present in a preparation is substantially purified. Typically, “purified” means that the protein is at least 85% pure, more preferably at least 95% pure, and most preferably at least 99% pure relative to the components with which the protein naturally occurs.

As used herein, an “antibody” refers to a protein functionally defined as a binding protein and structurally defined as comprising an amino acid sequence that is recognized by one of skill as being derived from the framework region of an immunoglobulin-encoding gene of an animal that produces antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

The term “antibody” as used herein includes antibody fragments that retain binding specificity. For example, there are a number of well characterized antibody fragments. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

Antibodies include dimers such as V_{H}-V_{L} dimers, V_{H} dimers, or V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment, such as a disulfide-stabilized Fv (dsFv). Other fragments can also be generated, including using recombinant techniques. The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). In some embodiments, antibodies include those that have been displayed on phage or generated by recombinant technology using vectors where the chains are secreted as soluble proteins, e.g., scFv, Fv, Fab, (Fab′)₂ or generated by recombinant technology using vectors where the chains are secreted as soluble proteins. Antibodies for use in the invention can also include diantibodies and miniantibodies.

Antibodies of the invention also include heavy chain dimers, such as antibodies from camelids. Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called VHH domains. Antibodies for use in the current invention include single domain antibodies (dAbs) and nanobodies (see, e.g., Cortez-Retamozo, et al., Cancer Res. 64:2853-2857, 2004).

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, for example, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29 (1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, “neutralizing antibody” refers to an antibody that binds to GM-CSF and prevents signaling by the GM-CSF receptor, or inhibits binding of GM-CSF to its receptor.

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule that confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in which the CDRs of a recipient human antibody are replaced by CDRs from a donor antibody. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci. USA 91: 969, 1994).

A “humaneered” antibody in the context of this invention refers to an engineered human antibody having a binding specificity of a reference antibody. A “humaneered” antibody for use in this invention has an immunoglobulin molecule that contains minimal sequence derived from non-human immunoglobulin. Typically, an antibody is “humaneered” by joining a DNA sequence encoding a binding specificity determinant (BSD) from the CDR3 region of the heavy chain of the reference antibody to human V_{H} segment sequence and a light chain CDR3 BSD from the reference antibody to a human V_{L} segment sequence. A “BSD” refers to a CDR3-FR4 region, or a portion of this region that mediates binding specificity. A binding specificity determinant therefore can be a CDR3-FR4, a CDR3, a minimal essential binding specificity determinant of a CDR3 (which refers to any region smaller than the CDR3 that confers binding specificity when present in the V region of an antibody), the D segment (with regard to a heavy chain region), or other regions of CDR3-FR4 that confer the binding specificity of a reference antibody. Methods for humaneering are provided in US patent application publication no. 20050255552 and US patent application publication no. 20060134098.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a heterologous protein will often refer to two or more subsequences that are not found in the same relationship to each other in nature.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequence at least two times the background and more typically more than 10 to 100 times background.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with GM-CSF protein and not with other proteins. This selection may be achieved by subtracting out antibodies that cross-react with other molecules.

As used herein, “ITP therapeutic agent” refers to an agent that when administered to a patient suffering from ITP, in a therapeutically effective dose, will cure, or at least partially arrest the symptoms of the disease and complications associated with the disease.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” in some embodiments denotes that a protein gives rise to essentially one band in an electrophoretic gel. Preferably, it means that the protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

### II. Introduction

The invention relates to methods of administering a GM-CSF antagonist for the treatment of patients diagnosed with ITP. The GM-CSF antagonists may include anti-GM-CSF antibodies, anti-GM-CSF receptor antibodies, or other inhibitors that prevent or reduce signaling that normally results from the binding of GM-CSF to its cognate receptor.

Antibodies, e.g., anti-GM-CSF or anti-GM-CSF receptor antibodies, suitable for use with the present invention may be monoclonal, polyclonal, chimeric, humanized, humaneered, or human. Other GM-CSF antagonists suitable for use with the present invention may include naturally occurring or synthetic ligands (or fragments thereof) that compete with GM-CSF for binding to the receptor, but do not result in signaling when bound to the receptor. Additional non-limiting GM-CSF antagonists may include polypeptides, nucleic acids, small molecules and the like that either partially or completely block signaling that would naturally result from the binding of GM-CSF to its receptor in the absence of the GM-CSF antagonist.

### III. Patients with ITP

ITP is a relatively common hematological disorder defined by low platelet count, normal bone marrow and the absence of other causes of thrombocytopenia. ITP can be diagnosed using standard clinical laboratory tests are used, including: urinalysis, CBC with differential, hematology, coagulation, serum chemistry (includes determining concentration of GM-CSF and soluble GM-CSF), surfactant D, erythrocyte sedimentation rate, and C-reactive protein.

Patients with chronic ITP are identified as at risk for bleeding if platelet the count is less than 30×10^{9/1} for those patients not receiving corticosteroids or less than 50×10⁹/L for those patients receiving corticosteroids.

Patient response to a therapy can be evaluated by monitoring any of the clinical parameters associated with ITP. Typically, a patient that exhibits a therapeutic response to treatment is determined by the presence of an increased platelet count above those thresholds that place the patient at risk for bleeding (30×10^{9/1} without corticosteroids and 50×10^{9/1} with corticosteroids) and increased clotting time or changes in the levels of other pharmacodynamic markers.

### IV. GM-CSF Antagonists

As noted above, the invention provides methods for treating ITP by administering a GM-CSF antagonist to a patient suffering from ITP. GM-CSF antagonists suitable for use in the invention selectively interfere with the induction of signaling by the GM-CSF receptor, e.g., by causing a reduction in the binding of GM-CSF to the receptor. Such antagonists may include antibodies that bind the GM-CSF receptor, antibodies that bind GM-CSF, and other proteins or small molecules that compete for binding of GM-CSF to its receptor or inhibit signaling that normally results from the binding of the ligand to the receptor.

In many embodiments, the GM-CSF antagonist used in the invention is a protein, e.g., an anti-GM-CSF antibody, an anti-GM-CSF receptor antibody, a soluble GM-CSF receptor, or a modified GM-CSF polypeptide that competes for binding with GM-CSF to a receptor, but is inactive. Such proteins are often produced using recombinant expression technology. Such methods are widely are widely known in the art. General molecular biology methods, including expression methods, can be found, e.g., in instruction manuals, such as, Sambrook and Russel (2001) Molecular Cloning: A laboratory manual 3rd ed. Cold Spring Harbor Laboratory Press; Current Protocols in Molecular Biology (2006) John Wiley and Sons ISBN: 0-471-50338-X.

A variety of prokaryotic and/or eukaryotic based protein expression systems may be employed to produce a GM-CSF antagonist protein. Many such systems are widely available from commercial suppliers. These include both prokaryotic and eukaryotic expression systems.

### GM-CSF Antibodies

In some embodiments, the GM-CSF antagonist is an antibody that binds GM-CSF or an antibody that binds to the GM-CSF receptor α or β subunit. The antibodies can be raised against GM-CSF (or GM-CSF receptor) proteins, or fragments, or produced recombinantly. Antibodies to GM-CSF for use in the invention can be neutralizing or can be non-neutralizing antibodies that bind GM-CSF and increase the rate of in vivo clearance of GM-CSF such that the GM-CSF level in the circulation is reduced. Often, the GM-CSF antibody is a neutralizing antibody.

Methods of preparing polyclonal antibodies are known to the skilled artisan (e.g., Harlow & Lane, Antibodies, A Laboratory manual (1988); Methods in Immunology). Polyclonal antibodies can be raised in a mammal by one or more injections of an immunizing agent and, if desired, an adjuvant. The immunizing agent includes a GM-CSF or GM-CSF receptor protein, or fragment thereof.

In some embodiment, a GM-CSF antibody for use in the invention is purified from human plasma. In such embodiments, the GM-CSF antibody is typically a polyclonal antibody that is isolated from other antibodies present in human plasma. Such an isolation procedure can be performed, e.g., using known techniques, such as affinity chromatography.

In some embodiments, the GM-CSF antagonist is a monoclonal antibody. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent, such as human GM-CSF, to elicit lymphocytes that produce or are capable of producing antibodies that will specifically bind to the immunizing agent. Alternatively, the lymphocytes may be immunized in vitro. The immunizing agent preferably includes human GM-CSF protein, fragments thereof, or fusion protein thereof.

Human monoclonal antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147 (1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments the anti-GM-CSF antibodies are chimeric or humanized monoclonal antibodies. As noted supra, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

An antibody that is employed in the invention can be in any format. For example, in some embodiments, the antibody can be a complete antibody including a constant region, e.g., a human constant region, or can be a fragment or derivative of a complete antibody, e.g., an Fd, a Fab, Fab′, F(ab′)₂, a scFv, an Fv fragment, or a single domain antibody, such as a nanobody or a camelid antibody. Such antibodies may additionally be recombinantly engineered by methods well known to persons of skill in the art. As noted above, such antibodies can be produced using known techniques.

In some embodiments of the invention, the antibody is additionally engineered to reduced immunogenicity, e.g., so that the antibody is suitable for repeat administration. Methods for generating antibodies with reduced immunogenicity include humanization/humaneering procedures and modification techniques such as de-immunization, in which an antibody is further engineered, e.g., in one or more framework regions, to remove T cell epitopes.

In some embodiments, the antibody is a humaneered antibody. A humaneered antibody is an engineered human antibody having a binding specificity of a reference antibody, obtained by joining a DNA sequence encoding a binding specificity determinant (BSD) from the CDR3 region of the heavy chain of the reference antibody to human VH segment sequence and a light chain CDR3 BSD from the reference antibody to a human VL segment sequence. Methods for humaneering are provided in US patent application publication no. 20050255552 and US patent application publication no. 20060134098.

An antibody can further be de-immunized to remove one or more predicted T-cell epitopes from the V-region of an antibody. Such procedures are described, for example, in WO 00/34317.

In some embodiments, the variable region is comprised of human V-gene sequences. For example, a variable region sequence can have at least 80% identity, or at least 85% identity, at least 90% identity, at least 95% identity, at least 96% identity, at least 97% identity, at least 98% identity, or at least 99% identity, or greater, with a human germ-line V-gene sequence.

An antibody used in the invention can include a human constant region. The constant region of the light chain may be a human kappa or lambda constant region. The heavy chain constant region is often a gamma chain constant region, for example, a gamma-1, gamma-2, gamma-3, or gamma-4 constant region.

In some embodiments, e.g., where the antibody is a fragment, the antibody can be conjugated to another molecule, e.g., to provide an extended half-life in vivo such as a polyethylene glycol (pegylation) or serum albumin. Examples of PEGylation of antibody fragments are provided in Knight et al (2004) Platelets 15: 409 (for abciximab); Pedley et al (1994) Br. J. Cancer 70: 1126 (for an anti-CEA antibody) Chapman et al (1999) Nature Biotech. 17: 780.

### Antibody Specificity

An antibody for use in the invention binds to GM-CSF or GM-CSF receptor. Any number of techniques can be used to determine antibody binding specificity. See, e.g., Harlow & Lane, Antibodies, A Laboratory Manual (1988) for a description of immunoassay formats and conditions that can be used to determine specific immunoreactivity of an antibody.

An exemplary antibody suitable for use with the present invention is c19/2. In some embodiments, a monoclonal antibody that competes for binding to the same epitope as c19/2, or that binds the same epitope as c19/2, is used. The ability of a particular antibody to recognize the same epitope as another antibody is typically determined by the ability of the first antibody to competitively inhibit binding of the second antibody to the antigen. Any of a number of competitive binding assays can be used to measure competition between two antibodies to the same antigen. For example, a sandwich ELISA assay can be used for this purpose. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### Epitope Mapping

In some embodiments of the invention, an antibody is employed that binds to the same epitope as a known antibody, e.g., c19/2. Method of mapping epitopes are well known in the art. For example, one approach to the localization of functionally active regions of human granulocyte-macrophage colony-stimulating factor (hGM-CSF) is to map the epitopes recognized by neutralizing anti-hGM-CSF monoclonal antibodies. For example, the epitope to which c19/2 (which has the same variable regions as the neutralizing antibody LMM102) binds has been defined using proteolytic fragments obtained by enzymic digestion of bacterially synthesized hGM-CSF (Dempsey, et al., Hybridoma 9:545-558, 1990). RP-HPLC fractionation of a tryptic digest resulted in the identification of an immunoreactive “tryptic core” peptide containing 66 amino acids (52% of the protein). Further digestion of this “tryptic core” with S. aureus V8 protease produced a unique immunoreactive hGM-CSF product comprising two peptides, residues 86-93 and 112-127, linked by a disulfide bond between residues 88 and 121. The individual peptides, were not recognized by the antibody.

### Determining Binding Affinity

In some embodiments, the antibodies suitable for use with the present invention have a high affinity binding for human GM-CSF or GM-CSF receptor. High affinity binding between an antibody and an antigen exists if the dissociation constant (K_{D}) of the antibody is <1 nM, and preferably <100 pM. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BLAcore™ 2000 instrument (Biacore AB, Freiburg, Germany) using CM5 sensor chips, as described by Krinner et al., (2007) Mol. Immunol. February; 44 (5):916-25. (Epub 2006 May 11)).

### Cell Proliferation Assay for Identifying Neutralizing Antibodies

In some embodiments, the GM-CSF antagonists are neutralizing antibodies to GM-CSF, or its receptor, which bind in a manner that interferes with the binding of GM-CSF. Neutralizing antibodies may be identified using any number of assays that assess GM-CSF function. For example, cell-based assays for GM-CSF receptor signaling, such as assays which determine the rate of proliferation of a GM-CSF-dependent cell line in response to a limiting amount of GM-CSF, are conveniently used. The human TF-1 cell line is suitable for use in such an assay. See, Krinner et al., (2007) Mol. Immunol. In some embodiments, the neutralizing antibodies of the invention inhibit GM-CSF-stimulated TF-1 cell proliferation by at least 50% when a GM-CSF concentration is used which stimulates 90% maximal TF-1 cell proliferation. In other embodiments, the neutralizing antibodies inhibit GM-CSF stimulated proliferation by at least 90%. Additional assays suitable for use in identifying neutralizing antibodies suitable for use with the present invention will be well known to persons of skill in the art.

### Exemplary Antibodies

Antibodies for use in the invention are known in the art and can be produced using routine techniques. Exemplary antibodies are described. It is understood that the exemplary antibodies can be engineered in accordance with the procedures known in the art and summarized herein to produce antibody fragments, chimeras, and the like by either chemical or recombinant technology.

An exemplary chimeric antibody suitable for use as a GM-CSF antagonist is c19/2. The c/19/2 antibody binds GM-CSF with a monovalent binding affinity of about 10 pM as determined by surface plasmon resonance analysis. SEQ ID NOs 1 and 2 show the heavy and light chain variable region sequence of c19/2 (e.g., WO03/068920). The CDRs, as defined according to Kabat, are:

| | |
|---|---|
| CDRH1 | DYNHI |
| CDRH2 | YIAPYSGGTGYNQEFKN |
| CDRH3 | RDRFPYYFDY |
| CDRL1 | KASQNVGSNVA |
| CDRL2 | SASYRSG |
| CDRL3 | QQFNRSPLT. |

The CDRs can also be determined using other well known definitions in the art, e.g., Chothia, international ImMunoGeneTics database (IMGT), and AbM.

The GM-CSF epitope recognized by c19/2 has been identified as a product that has two peptides, residues 86-93 and residues 112-127, linked by a disulfide bond between residues 88 and 121. The c19/2 antibody inhibits the GM-CSF-dependent proliferation of a human TF-1 leukemia cell line with an EC₅₀ of 30 pM when the cells are stimulated with 0.5 ng/ml GM-CSF.

An antibody for administration, such as c19/2, can be additionally humaneered. For example, the c19/2 antibody can be further engineered to contain human V gene segments.

Another exemplary neutralizing anti-GM-CSF antibody is the E10 antibody described in Li et al., (2006) PNAS 103 (10):3557-3562. E10 is an IgG class antibody that has an 870 pM binding affinity for GM-CSF. The antibody is specific for binding to human GM-CSF as shown in an ELISA assay, and shows strong neutralizing activity as assessed with a TF1 cell proliferation assay.

An additional exemplary neutralizing anti-GM-CSF antibody is the MT203 antibody described by Krinner et al., (Mol Immunol. 44:916-25, 2007; Epub 2006 May 112006). MT203 is an IgG1 class antibody that binds GM-CSF with picomolar affinity. The antibody shows potent inhibitory activity as assessed by TF-1 cell proliferation assay and its ability to block IL-8 production in U937 cells.

Additional antibodies suitable for use with the present invention will be known to persons of skill in the art.

GM-CSF antagonists that are anti-GM-CSF receptor antibodies can also be employed in the invention. Such GM-CSF antagonists include antibodies to the GM-CSF receptor alpha chain or beta chain. An anti-GM-CSF receptor antibody employed in the invention can be in any antibody format as explained above, e.g., intact, chimeric, monoclonal, polyclonal, antibody fragment, humanized, humaneered, and the like. Examples of anti-GM-CSF receptor antibodies, e.g., neutralizing, high-affinity antibodies, suitable for use in the invention are known (see, e.g., U.S. Pat. No. 5,747,032 and Nicola et al., Blood 82: 1724, 1993).

### Non-Antibody GM-CSF Antagonists

Other proteins which may interfere with the productive interaction of GM-CSF with its receptor include mutant GM-CSF proteins and secreted proteins comprising at least part of the extracellular portion of one or both of the GM-CSF receptor chains that bind to GM-CSF and compete with binding to cell-surface receptor. For example, a soluble GM-CSFR antagonist can be prepared by fusing the coding region of the sGM-CSFRalpha with the CH2-CH3 regions of murine IgG2a. An exemplary soluble GM-CSF receptor is described by Raines et al. (1991) Proc. Natl. Acad. Sci USA 88: 8203. An example of an GM-CSFRalpha-Fc fusion protein is provided, e.g., in Brown et al (1995) Blood 85: 1488. In some embodiments, the Fc component of such a fusion can be engineered to modulate binding, e.g., to increase binding, to the Fc receptor.

Other GM-CSF antagonist include GM-CSF mutants. For example, GM-CSF having a mutation of amino acid residue 21 of GM-CSF to Arginine or Lysine (E21R or E221K) described by Hercus et al. Proc. Natl. Acad. Sci USA 91:5838, 1994 has been shown to have in vivo activity in preventing dissemination of GM-CSF-dependent leukemia cells in mouse xenograft models (Iversen et al. Blood 90:4910, 1997). As appreciated by one of skill in the art, such antagonists can include conservatively modified variants of GM-CSF that have substitutions, such as the substitution noted at amino acid residue 21, or GM-CSF variants that have, e.g., amino acid analogs to prolong half-life.

In other embodiments, the GM-CSF antagonist is an “antibody mimetic” that targets and binds to the antigen in a manner similar to antibodies. Certain of these “antibody mimics” use non-immunoglobulin protein scaffolds as alternative protein frameworks for the variable regions of antibodies. For example, Ku et al. (Proc. Natl. Acad. Sci. U.S.A. 92 (14):6552-6556 (1995)) discloses an alternative to antibodies based on cytochrome b562 in which two of the loops of cytochrome b562 were randomized and selected for binding against bovine serum albumin. The individual mutants were found to bind selectively with BSA similarly with anti-BSA antibodies.

U.S. Pat. Nos. 6,818,418 and 7,115,396 disclose an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling may be employed in vitro that is similar to the process of affinity maturation of antibodies in vivo.

Beste et al. (Proc. Natl. Acad. Sci. U.S.A. 96 (5):1898-1903 (1999)) disclose an antibody mimic based on a lipocalin scaffold (Anticalin®). Lipocalins are composed of a β-barrel with four hypervariable loops at the terminus of the protein. The loops were subjected to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies. Thus, Anticalins® are small, single chain peptides, typically between 160 and 180 residues, which provides several advantages over antibodies, including decreased cost of production, increased stability in storage and decreased immunological reaction.

U.S. Pat. No. 5,770,380 discloses a synthetic antibody mimetic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol 49 (2):209-216 (2003)) describe a methodology for reducing antibodies into smaller peptidomimetics, they term “antibody like binding peptidomimetics” (ABiP) which may also be useful as an alternative to antibodies.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics). Accordingly, non-antibody GM-CSF antagonists can also include such compounds.

### V. Therapeutic Administration

The methods of the invention comprise administering a GM-CSF antagonist, (e.g., an anti-GM-CSF antibody) as a pharmaceutical composition to a patient having ITP in a therapeutically effective amount using a dosing regimen suitable for treatment of the disease. The composition can be formulated for use in a variety of drug delivery systems. One or more physiologically acceptable excipients or carriers can also be included in the compositions for proper formulation. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The GM-CSF antagonist for use in the methods of the invention is provided in a solution suitable for injection into the patient such as a sterile isotonic aqueous solution for injection. The GM-CSF antagonist is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The pharmaceutical compositions of the invention are administered to a patient with ITP in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include increased platelet count above 30×10^{9/1} and increased clotting time or changes in the levels of other pharmacodynamic markers. Amounts effective for this use will depend upon the severity of the disease and the general state of the patient's health, including other factors such as age, weight, gender, administration route, etc. Single or multiple administrations of the antagonist may be administered depending on the dosage and frequency as required and tolerated by the patient. In any event, the methods provide a sufficient quantity of GM-CSF antagonist to effectively treat the patient.

In another embodiment of the invention, the anti-GM-CSF antagonist used to treat a patient suffering from ITP is provided in combination therapy with another agent. Examples of other agents suitable for use in the invention are corticosteroids, such as methyl prednisolone or dexamethasone, IVIG, Anti-D or another antibody.

A. Administration

The invention provides methods for treatment of patients with ITP by administering a GM-CSF antagonist. In some embodiments, the GM-CSF antagonist is an antibody that is administered by injection or infusion through any suitable route including but not limited to intravenous, sub-cutaneous, intramuscular or intraperitoneal routes. In an exemplary embodiment, the antibody is stored at 10 mg/ml in sterile isotonic aqueous saline solution for injection at 4oC and is diluted in either 100 ml or 200 ml 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

B. Dosing

The dose of antagonist is chosen in order to provide effective therapy for the patient and is in the range of less than 0.1 mg/kg body weight to 25 mg/kg body weight or in the range 1 mg-2 g per patient. Preferably the dose is in the range 1-10 mg/kg or approximately 50 mg-1000 mg/patient. The dose may be repeated at an appropriate frequency which may be in the range once per day to once every three months, depending on the pharmacokinetics of the antagonists (e.g. half-life of the antibody in the circulation) and the pharmacodynamic response (e.g. the duration of the therapeutic effect of the antibody). In some embodiments where the antagonist is an antibody or modified antibody fragment, the in vivo half-life of between about 7 and about 25 days and antibody dosing is repeated between once per week and once every 3 months. In other embodiments, the antibody is administered approximately once per month.

### EXAMPLES

### Example 1

### Exemplary Humaneered Antibodies to GM-CSF

A panel of humaneered Fab′ molecules with the specificity of c19/2 were generated from epitope-focused human V-segment libraries as described in US patent application 20060134098.

Fab′ fragments were expressed from E. coli. Cells were grown in 2×YT medium to an OD600 of 0.6. Expression was induced using IPTG for 3 hours at 33° C. Assembled Fab′ was obtained from periplasmic fractions and purified by affinity chromatography using Streptococcal Protein G (HiTrap Protein G HP columns; GE Healthcare) according to standard methods. Fab's were eluted in pH 2.0 buffer, immediately adjusted to pH 7.0 and dialyzed against PBS pH7.4.

Binding kinetics were analyzed by Biacore 3000 surface plasmon resonance (SPR). Recombinant human GM-CSF antigen was biotinylated and immobilized on a streptavidin CM5 sensor chip. Fab samples were diluted to a starting concentration of 3 nM and run in a 3 fold dilution series. Assays were run in 10 mM HEPES, 150 mM NaCl, 0.1 mg/mL BSA and 0.005% p20 at pH 7.4 and 37° C. Each concentration was tested twice. Fab′ binding assays were run on two antigen density surfaces providing duplicate data sets. The mean affinity (K_{D}) for each of 6 humaneered anti-GM-CSF Fab clones, calculated using a 1:1 Langmuir binding model, is shown in Table 1.

Fabs were tested for GM-CSF neutralization using a TF-1 cell proliferation assay. GM-CSF-dependent proliferation of human TF-1 cells was measured after incubation for 4 days with 0.5 ng/ml GM-CSF using a MTS assay (Cell titer 96, Promega) to determine viable cells. All Fabs inhibited cell proliferation in this assay indicating that these are neutralizing antibodies. There is a good correlation between relative affinities of the anti-GM-CSF Fabs and EC₅₀ in the cell-based assay. Anti-GM-CSF antibodies with monovalent affinities in the range 18 pM-104 pM demonstrate effective neutralization of GM-CSF in the cell-based assay.

TABLE 1
Affinity of anti-GM-CSF Fabs determined by surface plasmon
resonance analysis in comparison with activity (EC₅₀) in
a GM-CSF dependent TF-1 cell proliferation assay

| | Monovalent | EC₅₀(pM) in |
|---|---|---|
| | binding affinity | TF-1 cell |
| | determined by | proliferation |
| Fab | SPR (pM) | assay |
| 94 | 18 | 165 |
| 104 | 19 | 239 |
| 77 | 29 | 404 |
| 92 | 58 | 539 |
| 42 | 104 | 3200 |
| 44 | 81 | 7000 |

### Example 2

### Clinical Protocol for Delivery of Anti-GM-CSF Antibody

An anti-GM-CSF antibody is stored at 10 mg/ml in sterile isotonic aqueous saline solution for injection at 4° C. and is diluted in either 100 ml or 200 ml 0.9% sodium chloride for injection prior to administration to the patient. The antibody is administered to a patient having ITP by intravenous infusion over the course of 1 hour at a dose of between 0.2 and 10 mg/kg.

The above examples are provided by way of illustration only and not by way of limitation. Those of skill in the art will readily recognize a variety of noncritical parameters that could be changed or modified to yield essentially similar results.

All publications, patent applications, accession numbers, and other references cited in this specification are herein incorporated by reference as if each individual publication or patent application were specifically and individually indicated to be incorporated by reference.

### Exemplary Sequences

SEQ ID NO 1: amino acid sequence for murine 19/2
heavy chain variable region
Met Glu Leu Ile Met Leu Phe Leu Leu Ser Gly Thr
Ala Gly Val His Ser Glu Val Gln Leu Gln Gln Ser
Gly Pro Glu Leu Val Lys Pro Gly Ala Ser Val Lys
Ile Ser Cys Lys Ala Ser Gly Tyr Thr Phe Thr Asp
Tyr Asn Ile His Trp Val Lys Gln Ser His Gly Lys
Ser Leu Asp Trp Ile Gly Tyr Ile Ala Pro Tyr Ser
Gly Gly Thr Gly Tyr Asn Gln Glu Phe Lys Asn Arg
Ala Thr Leu Thr Val Asp Lys Ser Ser Ser Thr Ala
Tyr Met Glu Leu Arg Ser Leu Thr Ser Asp Asp Ser
Ala Val Tyr Tyr Cys Ala Arg Arg Asp Arg Phe Pro
Tyr Tyr Phe Asp Tyr Trp Gly Gln Gly Thr Thr Leu
Arg Val Ser Ser Val Ser Gly Ser
SEQ ID NO 2: amino acid sequence for murine 19/2
light chain variable region
Met Gly Phe Lys Met Glu Ser Gln Ile Gln Val Phe
Val Tyr Met Leu Leu Trp Leu Ser Gly Val Asp Gly
Asp Ile Val Met Ile Gln Ser Gln Lys Phe Val Ser
Thr Ser Val Gly Asp Arg Val Asn Ile Thr Cys Lys
Ala Ser Gln Asn Val Gly Ser Asn Val Ala Trp Leu
Gln Gln Lys Pro Gly Gln Ser Pro Lys Thr Leu Ile
Tyr Ser Ala Ser Tyr Arg Ser Gly Arg Val Pro Asp
Arg Phe Thr Gly Ser Gly Ser Gly Thr Asp Phe Ile
Leu Thr Ile Thr Thr Val Gln Ser Glu Asp Leu Ala
Glu Tyr Phe Cys Gln Gln Phe Asn Arg Ser Pro Leu
Thr Phe Gly Ser Gly Thr Lys Leu Glu Leu Lys Arg
Ala Asp Ala Ala Pro Thr Val Ser Ile Phe Pro Pro
Ser Ser Lys Gly Glu Phe

What is claimed is:

1. A method for treating a patient suffering from idiopathic thrombocytopenia purpura (ITP), the method comprising administering a therapeutically effective amount of a purified GM-CSF antagonist to the patient in an amount sufficient to reduce the symptoms of ITP.

2. The method of claim 1, wherein the GM-CSF antagonist is an anti-GM-CSF antibody.

3. The method of claim 2, wherein the antibody is a polyclonal antibody.

4. The method of claim 2, wherein the antibody is a monoclonal antibody.

5. The method of claim 2, wherein the antibody is an antibody fragment that is a Fab, a Fab′, a F(ab′)₂, a scFv, or a dAB.

6. The method of claim 5, wherein the antibody fragment is conjugated to polyethylene glycol.

7. The method of claim 2, wherein the antibody has an affinity ranging from about 5 pM to about 50 pM.

8. The method of claim 2, wherein the antibody is a neutralizing antibody.

9. The method of claim 2, wherein the antibody is a recombinant or chimeric antibody.

10. The method of claim 2, wherein the antibody is a human antibody.

11. The method of claim 2, wherein the antibody comprises a human variable region.

12. The method of claim 2, wherein the antibody comprises a human light chain constant region.

13. The method of claim 2, wherein the antibody comprises a human heavy chain constant region.

14. The method of claim 13, wherein the human heavy chain constant region is a gamma chain.

15. The method of claim 2, wherein the antibody binds to the same epitope as chimeric 19/2.

16. The method of claim 2, wherein the antibody comprises the V_{H} and V_{L} regions of chimeric 19/2.

17. The method of claim 16, wherein the antibody comprises a human heavy chain constant region.

18. The method of claim 17, wherein the human heavy chain constant region is a gamma region.

19. The method of claim 2, wherein the antibody comprises the V_{H} region and V_{L} region CDR1, CDR2, and CDR3 of chimeric 19/2.

20. The method of claim 2, wherein the antibody comprises the V_{H} region CDR3 and V_{L} region CDR3 of chimeric 19/2.

21. The method of claim 1, further comprising administering a second ITP therapeutic agent selected from the group consisting of a corticosteroid, IVIG, and Anti-D.

22. The method of claim 1, wherein the GM-CSF antagonist is selected from the group consisting of an anti-GM-CSF receptor antibody; a soluble GM-CSF receptor; a cytochrome b562 antibody mimetic; an adnectin; a lipocalin scaffold antibody mimetic; a calixarene antibody mimetic; and an antibody like binding peptidomimetic.

23. A method for treating a patient suffering from idiopathic thrombocytopenia purpura (ITP), the method comprising administering a therapeutically effective amount of a an anti-GM-CSF antibody, wherein the anti-GM-CSF antibody comprises a humaneered Fab′ with the binding specificity of chimeric 19/2 and has an affinity ranging from about 5 to about 50 pM.


SEQUENCE LISTING (8 sequences)

SEQ ID NO: 1 - protein, 140 aa, Mus sp.
  murine 19/2 anti-hGM-CSF monoclonal antibody heavy chain variable region
MELIMLFLLSGTAGVHSEVQLQQSGPELVKPGASVKISCKASGYTFTDYNIHWVKQSHGKSLDWIGYIAPYSGGTGYNQEFKNRATLTVDKSSSTAYMELRSLTSDDSAVYYCARRDRFPYYFDYWGQGTTLRVSSVSGS

SEQ ID NO: 2 - protein, 150 aa, Mus sp.
  murine 19/2 anti-hGM-CSF monoclonal antibody light chain variable region
MGFKMESQIQVFVYMLLWLSGVDGDIVMIQSQKFVSTSVGDRVNITCKASQNVGSNVAWLQQKPGQSPKTLIYSASYRSGRVPDRFTGSGSGTDFILTITTVQSEDLAEYFCQQFNRSPLTFGSGTKLELKRADAAPTVSIFPPSSKGEF

SEQ ID NO: 3 - protein, 5 aa, Artificial Sequence
  chimeric c19/2 neutralizing anti-hGM-CSF antagonist monoclonal antibody CDRH1
DYNIH

SEQ ID NO: 4 - protein, 17 aa, Artificial Sequence
  chimeric c19/2 neutralizing anti-hGM-CSF antagonist monoclonal antibody CDRH2
YIAPYSGGTGYNQEFKN

SEQ ID NO: 5 - protein, 10 aa, Artificial Sequence
  chimeric c19/2 neutralizing anti-hGM-CSF antagonist monoclonal antibody CDRH3
RDRFPYYFDY

SEQ ID NO: 6 - protein, 11 aa, Artificial Sequence
  chimeric c19/2 neutralizing anti-hGM-CSF antagonist monoclonal antibody CDRL1
KASQNVGSNVA

SEQ ID NO: 7 - protein, 7 aa, Artificial Sequence
  chimeric c19/2 neutralizing anti-hGM-CSF antagonist monoclonal antibody CDRL2
SASYRSG

SEQ ID NO: 8 - protein, 9 aa, Artificial Sequence
  chimeric c19/2 neutralizing anti-hGM-CSF antagonist monoclonal antibody CDRL3
QQFNRSPLT